(19) United States
(12) Patent Application Publication
(10) Pub. No.: US 2002/0013255 A1
(43) Pub. Date: Jan. 31, 2002

(54) Anaerobically biodegradable perfume delivery system

(75) Inventors: Isao Noda, Fairfield, OH (US); Cynthia Lee Eddy, W. Harrison, IN (US); Pedro Antonio Rodriguez, Sanibel, FL (US)

Correspondence Address: THE PROCTER & GAMBLE COMPANY, PATENT DIVISION, IVORYDALE TECHNICAL CENTER - BOX 474, 5299 SPRING GROVE AVENUE, CINCINNATI, OH, 45217, US

(73) Assignee: The Procter & Gamble Co.

(21) Appl. No.: 09/885,694

(22) Filed: Jun. 20, 2001

Related U.S. Application Data
(60) Provisional application No. 60/216,093, filed on Jul. 6, 2000.

(51) Int. Cl. A61K 7/46

(52) U.S. Cl. 512/4; 510/108

(57) ABSTRACT

The present invention relates to a fragrance raw material delivery system comprising:

A) anaerobically biodegradable particles having:

i) an average particle size of from about 5 microns;

ii) a density of from about 0.9 g/cm³ to about 1.5 g/cm³; and

B) a fragrance or fragrance accord.

The fragrance delivery systems of the present invention are suitable for use in delivering a fragrance or fragrance accord to a situs, inter alia, fabric in a manner which sustains the delivery of the aesthetic elements while providing a completely biodegradable vehicle.

### CROSS REFERENCE

This Application claims priority to U.S. Provisional patent application Ser. No. 601216,093 filed Jul. 6, 2000.

### FIELD OF THE INVENTION

The present invention relates to delivery of perfumes or fragrances to a situs, preferably to fabric. The systems of the present invention comprise an anaerobically biodegradable particulate carrier, preferably a copolymer carrier and a fragrance or perfume.

### BACKGROUND OF THE INVENTION

Humans have used scents and fragrances since antiquity. Originally these aesthetically pleasing materials were commonly isolated in raw form as resins, gums or essential oils from natural sources, inter alia, the bark, roots, leaves and fruit of indigenous plants. These resins, gums, and oils were directly applied to the body or diluted with water or other solvent, including in some cases, wine. With the advent of modern chemistry, individual components responsible for the odor properties of these resins, gums and oils were isolated and subsequently characterized. Modern perfumery involves the artful compounding of fragrance materials to achieve novel fragrance compositions having defined “characteristics.” Many modern fragrances are no longer derived from natural sources but are synthesized by modern chemical methods as highly pure fragrance raw materials (FRM). These FRM's are currently formulated to produce fine perfumes, colognes, eau de toilettes, after-shave lotions, and other personal fragrance compositions. Those skilled in the art of preparing these fragrance-containing compositions have categorized fragrances into three types based on their relative volatility; top, middle, and base notes. In addition, fragrances are categorized by the odor they produce; some of these descriptors are broad and others are relatively specific. For example, “floral” is a term which connotes odors associated with flowers while the term “lilac” is more specific. Descriptors used by those skilled in the art of perfumes and fine fragrances are inter alia “rose,” “floral,” “green,” “citrus,” “spicy,” “honey,” and “musk.”

Top, middle, and base notes each serve a different purpose in the blending of fragrances and when properly formulated produce a “balanced fragrance” composition. Based on volatility, these notes are described by those skilled in the art as: the base notes having the most long lasting aroma; the middle notes, have a medium volatility; and the top notes are the most volatile. Key to successfully formulating a fragrance-containing composition is the precise balance between these three groups of materials producing a fragrance-containing composition that diffuses during its evaporation in a manner which has an aesthetic quality.

It is recognized by those skilled in the art that descriptors which relate to aesthetic perceptions such as “top,” “middle” and “base” notes are relative terms. A FRM categorized as a top note by one formulator usually has the identical classification among most other Perfumers. The same is true for the middle and base notes, however, occasionally one formulator may classify a given fragrance raw material as a middle note rather than a top note, or vice versa, but this fact does not diminish the utility of a given compound or its absolute identity. Top, middle and base notes are now combined in a reproducible manner to produce perfumes and fragrances for application to skin, fabric, or for release as air modifiers, all of which have unique and pleasant odor characteristics. Yet apart from this pleasant smell, a perfume or fragrance must meet a number of technical requirements. It must be sufficiently strong, it must be persistent, and it must retain its “essential character” throughout its period of evaporation. It is to these latter two requirements that the present invention is directed.

Due to the uneven rate of evaporation of the components which comprise a perfume or fragrance, the initial scent may be quite different than the aroma perceived several days, or even several hours later. This problem is inadequately solved in many different ways by the user. One method is to “load up” on the perfume initially and rely on the natural evaporation rate to diminish the fragrance to a suitable level several hours later when the desired effect is needed. Another method which is used is to continually renew the fragrance by reapplying small amounts of the perfume to the desired surface at short time intervals. Neither of these solutions is adequate to overcome the diminishing level of top and middle notes over time. In fact, base notes which are present over a protracted period by virtue of their low volatility, begin to accumulate with each “refreshing” of fragrance. In addition, these remedies are only suitable if the perfume or fragrance is directly applied to the skin or clothing of the user. This system fails for delivery of long term fragrances to targets, inter alia, fabric, wherein the fragrance is delivered by way of a unitized dose (most commonly via laundering).

Delivering benefits to fabric, inter alia, abrasion control, soil release benefits, softness, has been a goal of laundry formulators. The chief means for achieving a desired benefit is to deliver a material to fabric either reversibly or irreversibly. For example, permanent press benefits are delivered by reacting the fabric with one or more agents which insure a macroscopic structure to fabric. Those of ordinary skill in the art are aware of the advantages and drawbacks of any system which chemically alters the structure of the fiber itself. Remaining with the concept of permanent press, the term “durable” is used to connote a permanent press benefit which is of longer duration than state of the art or “normal” conditions. These terms connote that benefits or properties which are not inherent in the fabric itself, are at best finite in duration.

Soil release has been obtained by deposition of molecules onto fabric. The molecules are typically very similar to the molecular structure of the fabric on which they are most effect, for example, most soil release agents (SPA's) are terephthalate copolymers which are similar to polyester fabric. These agents work well only like polyester fabrics but are marginal or ineffective on cotton. Many other examples exist wherein the formulator attempts to provide a top layer to the fabric which are molecules of similar structure but which provide a property which enhances the fabric or which are permanently bound to said fabric and which either enhance the fabric (i.e. feel, lubricity, strength) or which modify the fabric itself (i.e., permanent press, fire retardance).

There exists a long felt need in the art for a system which can deliver fragrances, either singly or in the form of accords, to a situs, inter alia, fabric, which sustains the level of fragrance and which does not involve the concomitant loss of fragrance raw materials due to inadequate initial delivery. There exists a long felt need in the art for a system which is universal to all fabric types and to all fabric laundry conditions and which does not alter the structure of fabric, rely upon the properties of the fabric material, nor preparation of the fabric by the manufacturer. There is also a long felt need for a delivery system which is universal to fabric, but one which is environmentally compatible in that the system is anaerobically biodegradable.

### SUMMARY OF THE INVENTION

The present invention meets the aforementioned needs in that it has been surprisingly discovered that fragrances, accords, pro-fragrances, and pro-accords can be delivered to a situs, preferably to fabric, via a system which does not result in the substantial loss of fragrance raw materials during delivery, and which releases the fragrance ingredients in a controllable manner. In addition, and most importantly, the delivery systems of the present invention afford the use of materials which are readily formed and which are universally compatible to all fabric type and fabric weaves. The co-polymers which comprise the present invention are also anaerobically biodegradable materials. The advantage provided by this combination provides the formulator with a unique means for solving the problems which relate to fragrance aesthetics and the delivery thereof.

The co-polymers which comprise the embodiments of the present invention are environmentally compatible, anaerobically biodegradable materials which provide an advantage over conventional fragrance delivery systems.

The first aspect of the present invention relates to a fragrance raw material delivery system comprising:

A) particles having:

i) an average particle size sufficient to become entrapped on fabric, preferably from about 5μ (microns), more preferably from about 10 μ, yet more preferably from about 35μ, most preferably from about 40μ to about 300μ, preferably to about 200μ, more preferably to about 100 μ, most preferably to about 60μ;

ii) a density of from about 0.9 g/cm to about 1.5 g/cm³, preferably about 1 g/cm³; and

B) a fragrance or fragrance accord.

The present invention further relates to delivery of fragrance raw material comprising particles by way of entrapment or entrainment of said particle onto fabric.

The present invention relates to a method for delivering fragrance raw materials to fabric, said method comprising the step of applying a single fragrance raw material to the anaerobically biodegradable particles of the present invention, and then combining two or more individually “dosed” particles to form a perfume or fragrance accord.

The present invention also relates to compositions which comprise the fragrance delivery systems of the present invention. Said compositions are used to aesthetically enhance articles, inter alia, clothing.

The present invention also relates to laundry detergent compositions comprising the fragrance delivery systems of the present invention and to compositions which are used to refresh fabric.

A further aspect of the present invention relates to methods for delivering a fragrance or fragrance accord to a situs by contacting the material to be aesthetically enhanced with a composition which comprises the fragrance delivery system of the present invention.

These and other objects, features and advantages will become apparent to those of ordinary skill in the art from a reading of the following detailed description and the appended claims. All percentages, ratios and proportions herein are by weight, unless otherwise specified. All copolymer compositions defined as mole percent (mol %). Particle sizes are defined as the number average particle diameter. Polymer molecular weights are defined herein as the weight average. All temperatures are in degrees Celsius (°C) unless otherwise specified. All documents cited are in relevant part, incorporated herein by reference.

### DETAILED DESCRIPTION OF THE INVENTION

The present invention relates to a system for delivering fragrance raw materials to a situs. The preferred situs is an irregular surface having pores, openings, or other interstices. A non-limiting example of a preferred situs is fabric which comprises clothing, draperies, towels, linen, and the like.

Those of ordinary skill in the art recognize that fabric may comprise one material, inter alia, cotton, rayon, wool, or may be a blend of fabric, inter alia, a cotton-synthetic mixture. The artisan, to deliver any benefit to fabric, has commonly used one of several means. Like material will bind to like material. Using this premise, the formulator has employed electrostatic attraction, hydrogen bonding, and other properties related to substantivity to deposit benefit providing compounds or compositions to fabric surfaces. In some instances, these methods have proved effective in providing the consumer with a noticeable benefit, inter alia, fabric anti-static vis à vis quaternary ammonium compounds.

Perfumes, fragrances, accords, and fragrance raw material provide a problem which is not solvable by a fabric modification process or fabric compatibility change. Fragrance raw materials elicit an aesthetic response because of their chemical structure and change to which would modify or obliterate the property altogether. Therefore, fragrance materials must be delivered and released with minimal chemical or physical change. This has been accomplished by the formation of pro-fragrances or pro-perfumes which is not the subject of the present invention.

Unaltered fragrance raw materials have been delivered to fabric by a variety of carriers, inter alia, cyclodextrins, zeolites, amorphous silicates, micro-encapsulates all of which are limited as compared to the present invention. Each of these prior art delivery means relates to molecular delivery of fragrance raw materials in contrast to the delivery systems of the present invention. Unlike the particles which comprise the delivery systems of the present invention, a particular cyclodextrin which is loaded with a particular fragrance raw material will not adhere equally to all fabric types, nor will the cyclodextrin be delivered as efficiently to fabric as the particles of the present invention. Cyclodextrins form an inclusion complex with the fragrance raw material and the ability of a cyclodextrin to deliver a fragrance is based on the ability of the cyclodextrin to adhere to a specific type of fabric. This is not true for the particles of the present invention which are not fabric-type dependent. Efficiency and the universal means of deploying the systems of the present invention set the present invention apart. The particles of the present invention are entrained upon the fabric and are therefore not limited in scope as are the prior art fabric-type dependent delivery modes.

The delivery systems of the present invention rely on the size of the particles and not on an inherent property of the fabric, perfume ingredient, etc. for delivery of the fragrance benefit. Manufacturers which produce fabric rely on machinery and looms which produce a product having relatively uniform pore sizes. The particles of the present invention have a size which is sufficient to be entrapped or entrained by fabric thereby relying on no other fabric property. This system provides a surprisingly universal means for delivering a perfume based aesthetic benefit to fabric.

The present invention provides the artisan whose endeavor it is to increase or enhance the relative aesthetics of clothing, furniture, etc., with a means for delivering a perfume or fragrance which is enduring and which serves a long felt need. The nature of the delivery system allows the formulator to provide a controlled release of the fragrance or perfume thereby avoiding the differential levels of fragrance levels which are manifest in the current delivery methods. For example, when fragrances are initially applied by spraying or misting with liquid fragrances, the initial level is very strong. Due to evaporation, the level of fragrance soon diminishes to a level which is un noticed. Thus the differential evaporation of accords (mixtures of fragrance raw materials) not only provides a diminution of fragrance levels but of accord composition.

The copolymers which comprise the particles of the present invention are anaerobically biodegradable. For the purposes of the present invention the term “anaerobically biodegradable” is defined herein as “the ability of a particle to be converted by microbes to the lowest metabolic constituent endpoints, namely, water, carbon dioxide, and methane, said metabolic endpoints are achievable under reduced or depleted oxygen conditions.” The particles of the present invention can be degraded to water, carbon dioxide, and methane under the anaerobic conditions which may sometimes characterize laundry or other aqueous based effluent. Standing water, inter alia, ponds, holding tanks, run-offs, may comprise water which is insufficiently aerated to promote or sustain aerobiosis. Under these conditions, the copolymers which comprise the particles of the present invention are completely biodegradable. This ability to anaerobically biodegrade distinguishes the copolymers which comprise the particles of the present invention from other particles which are comprised of only aerobically biodegradable polymers, conventional organic polymers or copolymers or inorganic carriers, inter alia, zeolites, amorphous silicates.

The ability to degrade under anaerobic conditions provides the formulator with distinct marketing, cost, and safety advantages over the prior art particles described herein above. Environmental concerns, especially in geographies wherein ground water tables co-mix with effluent run-off or collection, are abated by the use of the particles of the present invention.

The systems of the present invention deliver fragrance raw materials in a manner which fill a niche or blind spot in the manner in which perfumes and fragrances are delivered and which provides a surprisingly effective result. The particles of the present invention serve to “carry” or “deliver” fragrances to a situs. The particles of the present invention in addition to the biodegradable advantages, deliver aesthetic benefits in a surprising manner. The particles of the present invention are copolymers which are comprised of semi-crystalline, “plastic” materials. Providing a rigidity necessary to withstand processing, delivery, and mechanical stresses, the particles comprise a hydrophobic portion into which fragrance raw materials are easily absorbed and released therefrom while providing a hydrophobic barrier to premature extraction by the laundry liquor. In addition, each particle has the capacity to transfer from 2% to 50% of its mass in fragrance raw materials.

The particles are delivered to a situs, preferably fabric, by means of entrapment or entrainment. This is accomplished by providing a particle having a size greater than the average porosity of fabric. In addition, the particles are carried and sustained by water without the water leaching out or extracting the fragrance raw materials. For example, residual water from the application of said particles, for example, laundry liquor, can help sustain the particle onto fabric.

The preferred particles of the present invention have their average diameter size defined in microns, the term “microns” and the symbol “μ” being used herein interchangeably and standing for the same scalar value. The particle of the present invention have a size sufficient to become entrapped, that is a size large enough to become entrapped by fabric, preferably the particles have an average size of from about 5 μ (microns), more preferably from about 10 μ, yet more preferably from about 35μ, most preferably from about 40μ to about 300μ, preferably to about 200μ, more preferably to about 100μ, most preferably to about 60 μ. Other particle parameters and characteristics will be laid forth herein below.

One key embodiment of the present invention relates to the sustained release of fragrance on fabric wherein said particles are delivered via fully formulated laundry detergent compositions. Typically fragrances are delivered to fabric “through the wash.” However, this method fails for several reasons: the solubility of the fragrance raw materials for water result in much of the fragrance being carried away during the wash, the volatility of FRM's during automatic clothes drying. Each of these intrusions not only results in a diminished level of ingredients but a loss of fragrance “balance.” The present invention provides a carrier component which insures a high delivery rate of fragrance raw materials as well as the integrity of the fragrance character itself. Also, the carrier is biodegradable so there is no worry when large amounts of material is used and released to the environment. This is especially true if the systems of the present invention are employed by a manufacturer who wishes to provide an initial aesthetic fragrance benefit to an article of manufacture.

Particles

The particles which comprise the fragrance delivery systems of the present invention comprise an polymer or copolymer which can suitably absorb and deliver the fragrance benefits described herein to fabric. The polymers or copolymers can comprise an monomeric units provided the resulting polymer has the ability to carry one or more fragrance raw materials to a fabric and release said materials once delivered.

The anaerobically biodegradable perfume delivery particles of the present invention are comprised of:

i) lower alkyl substituted units having the formula:

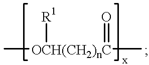

wherein R¹ is hydrogen, C₁-C₂ alkyl, and mixtures thereof; preferably R¹ is methyl or ethyl, more preferably methyl. The index n is 1 or 2; preferably 1.

ii) higher alkyl substituted units having the formula:

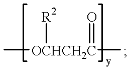

wherein R² is C₃-C₁₉ alkyl, C₃-C₁₉ alkenyl, and mixtures thereof; preferably C₄-C₁₅ alkyl, more preferably C₄-C₈ alkyl. Another higher alkyl substituted unit suitable for use in the present invention has the formula:

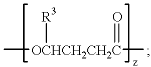

wherein R³ is C₃-C₁₉ alkyl, C₃-C₁₉ alkenyl, and mixtures thereof; preferably C₄-C₈ alkyl. For the purposes of the present invention R² is not equal to R³. The indices x, y, and z have values such that the sum of x+y or the sum of x+y+z produces a copolymer having a weight average molecular weight of from about 500 daltons, preferably from about 1000 daltons, more preferably from about 5,000 daltons, most preferably from about 10,000 daltons to about 5,000,000 daltons, preferably to about 1,000,000 daltons, more preferably to about 500,000 daltons, most preferably to about 100,000 daltons

The anaerobically biodegradable copolymers of the present invention comprise mixtures of lower alkyl substituted units and higher alkyl substituted units such that at least about 50% of said copolymer comprises lower alkyl substituted units. The copolymers of the present invention are random copolymers.

Preferred lower alkyl substituted monomer units according to the present invention include 3-hydroxyvalerate, 3-hydroxybutyrate, and mixtures thereof.

In another embodiment of the present invention 3-hydroxypropionate monomer units are included in the admixture of lower alkyl substituted monomer units.

The anaerobically biodegradable copolymers of the present invention are formed into carrier particles by any conventional means, said particles having an average size range from about 5μ (microns), preferably from about 10μ, more preferably from about 35μ, most preferably from about 40μ to about 300μ, preferably to about 200 μ, more preferably to about 100μ, most preferably to about 60μ. Additional processing advantages are described in U.S. Pat. No. 5,899,339 Noda, issued May 4, 1999; and U.S. Pat. No. 5,918,747 Noda, issued Jul. 6, 1999 both of which are incorporated herein by reference.

The anaerobically biodegradable copolymers of the present invention have a melt temperature Tm, of from about 30° C., preferably from about 60° C., more preferably from about 90° C. to about 160° C., preferably to about 140° C., more preferably to about 120° C.

A detailed description of preferred anaerobically biodegradable copolymers according to the present invention is contained in U.S. RE 36,548 Noda, which is a re-issue of U.S. Pat. No. 5,602,227 Noda, issued Feb. 11, 1997; U.S. Pat. No. 5,502,116 Noda, issued Mar. 26, 1996 both of which are included herein by reference. A detailed description of methods for making biodegradable copolymers according to the present invention is described in U.S. Pat. No. 5,648,452 Schechtman et al., issued Jun. 15, 1997 included herein by reference.

The particle once formed and once having the fragrance raw material carrier properties suitable for executing delivery of the fragrance or fragrance accord, does not need to be further adjusted or adapted by the formulator. Because the particles of the present invention are entrapped or entrained onto the surface of the situs, inter alia, fabric, formulation and particle delivery issues relating to substantivity, affinity, or other variables important to insuring deposition and adherence of the particles to the situs, can be abrogated by the formulator. Unlike other carrier particles, inter alia, zeolites, amorphous clays and silicates, the particles of the present invention are delivered to the situs where they remain until removed by dislodging, i.e. by laundering with water.

Fragrance Component

The second essential ingredient according to the present invention relates to fragrances and perfumes which are delivered via the systems of the present invention. The compounds which comprise the fragrance component consist of:

i) fragrance raw materials, inter alia, aldehydes, ketones, alcohols, esters, ethers, nitrites, terpenes, which are the aesthetic odor constituents of fragrances and perfumes;

ii) pro-fragrance raw materials, inter alia, orthoesters, acetals, ketals, which are capable of breaking down over time to controllably release one or more fragrance raw materials; and

iii) optionally, carriers, diluents, and the like which may serve to assist in delivering, stabilizing, or otherwise insuring fragrance integrity.

For the purposes of the present invention the terms “perfume” and “fragrance” are essentially synonymous and are used collectively or interchangeably throughout the present specification and are taken to mean the more concentrated forms of fragrance-containing compositions. Aspects of the present invention which apply to “perfumes” will therefore apply equally to “fragrances” and vice versa. The present invention relates to perfume and fragrance delivery compositions having scent or odor longevity benefits wherein these benefits are achieved by controllably releasing the fragrance components that comprise the perfume or fragrance.

Perfumes and fragrances typically comprise components which react with human olfactory sites resulting in what is known as “fragrance.” Typical molecules which comprise perfume fragrances are linear and cyclic alkenes (i.e., terpenes), primary, secondary and tertiary alcohols, nitrites, ethers, saturated and unsaturated aldehydes, esters, ketones, and mixtures thereof. Each of these perfume fragrances can be classified according to its volatility into one of three categories; “top note,” “middle note,” and “base note.”

For the purposes of the present invention “top note” fragrances are defined as “fragrances having a high vapor pressure, and when applied to a paper sachet, vaporization takes place within 2 hours, and no scent remains; essentially, the initial impression of the perfume formulation is provided by top notes.”

For the purposes of the present invention “middle note” fragrances are defined as “fragrances having a medium vapor pressure, and when applied to a paper sachet, the scent remains from about 2 to about 6 hours; essentially, middle notes provide the skeleton of the perfume formulation.”

For the purposes of the present invention “base note” fragrances are defined as “fragrances having a low vapor pressure and high retentivity, and when applied to a paper sachet, the scent remains for more than about 6 hours; essentially, base notes provide the characteristic of the perfume formulation.

The terms “top note,” “middle note,” and “base note” are well recognized by those skilled in the art of fragrance-containing compositions. However, reference to a specific fragrance raw material as a “top note” within the present invention does mean that others skilled in the art of fragrance-containing compositions may not categorized the same ingredient as a “middle note.” The same applies to fragrance raw materials referred to as “middle notes” and “base notes.”

Fragrance Raw Materials and Accords

Mixtures of fragrance materials are known by those skilled in the art of fragrances and perfumes as “accords.” The term “accord” as used herein is defined as “a mixture of two or more fragrance raw materials which are artfully combined to impart a pleasurable scent, odor, essence, or fragrance characteristic.” For the purposes of the present invention “fragrance raw materials” are herein defined as compounds having a molecular weight of at least 100 g/mol and which are useful in imparting an odor, fragrance, essence, or scent either alone or in combination with other “fragrance raw materials.”

Typically “fragrance raw materials” comprise inter alia alcohols, ketones, aldehydes, esters, ethers, nitrites, and alkenes such as terpenes. A listing of common “fragrance raw materials” can be found in various reference sources, for example, “Perfume and Flavor Chemicals,” Vols. I and II; Steffen Arctander Allured Pub. Co. (1994) and “Perfumes: Art, Science and Technology”; Mtiller, P. M. and Lamparsky, D., Blackie Academic and Professional (1994) both incorporated herein by reference.

Non-limiting examples of alcohols which are suitable for use as fragrance raw materials or which are suitably released by the hydrolysis of the pro-fragrances or pro-accords of the present invention include 2,4-dimethyl-3-cyclohexene-1-methanol (Floralol), 2,4-dimethyl cyclohexane methanol (Dihydrofloralol), 5,6-dimethyl-1-methylethenyl-bicyclo[2.2.1] hept-5-ene-2-methanol (Arbozol), 2,4,6-trimethyl-3-cyclohexene-1-methanol (Isocyclogeraniol), 4-(1-methylethyl)cyclohexanemethanol (Mayol), α-3,3-trimethyl-2-norborane 1,1-dimethyl-1-(4-methylcyclohex-3-enyl)methanol, 2-phenylethanol, 2-cyclohexyl ethanol, 2-(o-methylphenyl)-ethanol, 2-(m-methylphenyl)ethanol, 2-(p-methylphenyl)ethanol, 6,6-dimethylbicyclo-[3.1.1]hept-2-ene-2-ethanol (nopol), 2-(4-methylphenoxy)ethanol, 3,3-dimethyl-Δ²-β-norbo ethanol, 2-methyl-2-cyclohexyl-ethanol, 1-(4-isopropylcyclohexyl)-ethanol, 1-phenylethanol, 1,1-dimethyl-2-phenyl-ethanol, 1,1-dimethyl-2-(4-methyl-phenyl)ethanol, 1-phenylpropanol, 3-phenylpropanol, 2-phenylpropanol (Hydrotropic Alcohol), 2-(cyclododecyl)propan-1-ol (Hydroxy-ambran), 2,2-dimethyl-3-(3-methylphenyl)propan-1-ol (Majantol), 2-methyl-3-phenylpropanol, 3-phenyl-2-propen-1-ol (cinnamyl alcohol), 2-methyl-3-phenyl-2-propen-1-ol (methyl-cinnamyl alcohol), (x-n-pentyl-3-phenyl-2-propen-1-ol ((x-amyl-cinnamyl alcohol), ethyl-3-hydroxy-3-phenyl propionate, 2-(4-methylphenyl)-2-propanol, 3-methylbutanol, 3-(4-methylcyclohex-3-ene)butanol, 2-methyl-4-(2,2,3-trimethyl-3-cyclopenten-1-yl)butanol, 2-ethyl-4-(2,2,3-trimethylcyclopent-3-enyl)-2-buten-1-ol, 3-methyl-2-buten-1-ol, 2-methyl-4(2,2,3-trimethyl-3-cyclopenten-1-yl)-2-buten-1-ol, 3-hydroxy-2-butanone, ethyl 3-hydroxybutyrate, 4-phenyl-3-buten-2-ol, 2-methyl-4-phenylbutan-2-ol, 4-(4-hydroxy-phenyl)butan-2-one, 4-(4-hydroxy-3-methoxyphenyl)butan-2-one, cis-3-pentenol, 3-methyl-pentanol, 3-methyl-3-penten-1-ol, 2-methyl-4-phenylpentanol (Pamplefleur), 3-methyl-5-phenylpentanol (Phenoxanol), 2-methyl-5-phenylpentanol, 2-methyl-5-(2,3-dimethyltricyclo[2.2.1.0(2,6)]hept-3-yl)-2-penten-1-ol (santalol), 4-methyl-1-phenyl-2-pentanol, (1 -methyl-bicyclo[2.1.1]hepten-2-yl)-2-methylpent-1-en-3-ol, 3-methyl-l-phenylpentan-3-ol, 1 ,2dimethyl-3-( 1 -methylethenyl) cyclopentan-1-ol, 2-isopropyl-5-methyl-2-hexenol, cis-3-hexen-1-ol, trans-2-hexen-1-ol, 2-isoproenyl-4-methyl-4-hexen-1-ol (Lavandulol), 2-ethyl-2-prenyl-3-hexenol, 1-hydroxymethyl-4-iso-propenyl-1-cyclohexene (Dihydrocuminyl alcohol), 1-methyl-4-isopropenylcyclohex-6-en-2-ol (carvenol), 6-methyl-3-isopropenylcyclohexan-1-ol, 1 -methyl-4-iso-propenylcyclohexan-3-ol, 4-isopropyl-1-methylcyclohexan-3-ol, 4-tert-butylcyclo-hexanol, 2-tert-butylcyclo-hexanol, 2-tert-butyl-4-methylcyclohexanol, 4-isopropyl-cyclohexanol, 4-methyl-1-(1-methylethyl)-3-cyclohexen-1-ol, 2-(5,6,6-trimethyl-2-norbomyl)cyclohexanol, isobomyl-cyclohexanol, 3,3,5-trimethylcyclohexanol, 1-methyl-4-isopropylcyclohexan-3-ol, 1,2-dimethyl-3-(1-methylethyl)cyclohexan-1-ol, heptanol, 2,4-dimethylheptan-1-ol, 2,4-dimethyl-2,6-heptandienol, 6,6-dimethyl-2-oxymethylbicyclo [3.1.1]hept-2-ene (myrtenol), 4-methyl-2,4-heptadien-1-ol, 3,4,5,6,6-pentamethyl-2-heptanol, 3,6-dimethyl-3-vinyl-5-hepten-2-ol, 6,6-dimethy-3-hydroxy-2-methylenebicyclo[3.1.l]heptane, 1,7,7-trimethyl-bicyclo[2.2. l]heptan-2-ol, 2, 6-dimethylheptan-2-ol, 2,6,6-trimethylbicyclo[1.3.3]heptan-2-ol, octanol, 2-octenol, 2-methyloctan-2-ol, 2-methyl-6-methylene-7-octen-2-ol (myrcenol), 7-methyloctan-1-ol, 3,7-dimethyl-6-octenol, 3,7-dimethyl-7-octenol, 3,7-dimethyl-6-octen-1-ol (citronellol), 3,7-dimethyl-2,6-octadien-1-ol (geraniol), 3,7-dimethyl-2,6-octadien-1-ol (nerol), 3,7-dimethyl-1,6-octadien-3-ol (linalool), 3,7-dimethyloctan-1-ol (pelargol), 3,7-dimethyloctan-3-ol (tetrahydrolinalool), 2,4-octadien-1-ol, 3,7-dimethyl-6-octen-3-ol, 2,6-dimethyl-7-octen-2-ol, 2,6-dimethyl-5,7-octadien-2-ol, 4,7-dimethyl-4-vinyl-6-octen-3-ol, 3-methyloctan-3-ol, 2,6-dimethyloctan-2-ol, 2,6-dimethyloctan-3-ol, 3,6-dimethyloctan-3-ol, 2,6-dimethyl-7-octen-2-ol, 2,6-dimethyl-3,5-octadien-2-ol (muguol), 3-methyl-l-octen-3-ol, 7-hydroxy-3,7-dimethyl-octanal, 3-nonanol, 2,6-nonadien-1-ol, cis-6-nonen-1-ol, 6,8-dimethylnonan-2-ol, 3-(hydroxymethyl)-2-nonanone, 2-nonen-1-ol, 2,4-nonadien-1-ol, 3,7-dimethyl-1,6-nonadien-3-ol, decanol, 9-decenol, 2-benzyl-m-dioxa-5-ol, 2-decen-1-ol, 2,4-decadien-l-ol, 4-methyl-3-decen-5-ol, 3,7,9-trimethyl-1,6-decadien-3-ol (isobutyllinallol), undecanol, 2-undecen-1-ol, 10-undecen-1-ol, 2-dodecen-1-ol, 2,4-dodecadien-1-ol, 2,7,11-trimethyl-2,6,10-dodecatrien-1-ol (farnesol), 3,7,11-trimethyl-1,6,10,-dodecatrien-3-ol, 3,7,11,15-tetramethylhexadec-2-en-1-ol (phytol), 3,7,11,15-tetramethylhexadecl-en-3-ol (isophytol), benzyl alcohol, p-methoxy benzyl alcohol (anisyl alcohol), para-cymen-7-ol (cuminyl alcohol), 4-methyl benzyl alcohol, 3,4-methylenedioxy benzyl alcohol, methyl salicylate, benzyl salicylate, cis-3-hexenyl salicylate, n-pentyl salicylate, 2-phenylethyl salicylate, n-hexyl salicylate, 2-methyl-S-isopropylphenol, 4-ethyl-2-methoxyphenol, 4-allyl-2-methoxyphenol (eugenol), 2-methoxy-4-(1-propenyl)phenol (isoeugenol), 4-allyl-2,6-dimethoxy-phenol, 4-tert-butylphenol, 2-ethoxy-4-methylphenol, 2-methyl4-vinylphenol, 2-isopropyl-5-methylphenol (thymol), pentyl-ortho-hydroxy benzoate, ethyl 2-hydroxy-benzoate, methyl 2,4-dihydroxy-3,6-dimethylbenzoate, 3-hydroxy-5-methoxy-1-methylbenzene, 2-tert-butyl-4-methyl-1-hydroxybenzene, 1-ethoxy-2-hydroxy-4-propenylbenzene, 4-hydrozytoluene, 4-hydroxy-3-methoxybenzaldehyde, 2-ethoxy-4-hydroxybenzaldehyde, decahydro-2-naphthol, 2,5,5-trimethyl-octahydro-2-naphthol, 1,3,3-trimethyl-2-norbornanol (fenchol), 3a,4,5,6,7,7a-hexahydro-2,4-dimethyl-4, 7-methano-1H-inden-5-ol, 3a,4,5,6,7,7a-hexahydro-3,4-dimethyl-4,7-methano-1H-inden-5-ol, 2-methyl-2-vinyl-5-(1-hydroxy-1-methylethyl)tetrahydrofuran, β-caryophyllene alcohol, and mixtures thereof.

Non-limiting examples of esters which are suitable for use as fragrance raw materials or which are suitably released by the hydrolysis of the pro-fragrances or pro-accords of the present invention include geranyl formate, citronellyl formate, phenylethyl formate, phenoxyethyl formate, trans-2-hexenyl formate, cis-3-hexenyl formate, cis-6-nonenyl formate, 9-decenyl formate, 3,5,5-trimethylhexyl formate, 3-methyl-5-phenyl-pentanyl formate, 6-methylheptan-2-yl formate, 4-(2,2,6-trimethyl-2-cyclohexen-1-yl)-3-buten-2-yl formate, 3-methyl-5-(2,2,3-trimethyl-3-cyclopenten-1-yl)4-penten-2-yl formate, 4-isopropylcyclohexyleth-2-yl formate, 6,8-dimethylnonan-2-yl formate, decahydro-β-naphthyl formate, 4-isopropylcyclohexylmethyl formate, linalyl formate, lavandulyl formate, citronellyl formate, α-terpinyl formate, nopyl formate, isobornyl formate, bornyl formate, isobornyl formate, guaiyl formate, 2-tert-butylyclohexyl formate, 4-tert-butylcyclohexyl formate, decahydro-β-naphthyl formate, menthyl formate, p-menthanyl formate, neryl formate, cinnamyl formate, ethyl acetate, butyl acetate, isoamyl acetate, hexyl acetate, 3,5,5-trimethylhexyl acetate, geranyl acetate, citronellyl acetate, phenylethyl acetate, phenoxyethyl acetate, trans-2-hexenyl acetate, cis-3-hexenyl acetate, cis-6-nonenyl acetate, 9-decenyl acetate, 3-methyl-5-phenylpentanyl acetate, 6-methyl-heptan-2-yl acetate, 4-(2,2,6-trimethyl-2-cyclohexen-1-yl)-3-buten-2-yl acetate, 3-methyl-5-(2,2, 3-trimethyl-3-cyclopenten-1-yl)-4-penten-2-yl acetate, decahydro-o-naphthyl acetate, menthyl acetate, benzyl acetate, 4-isopropylcyclohexyleth-2-yl acetate, 6,8-dimethylnonan-2-yl acetate, 1-phenylethyl acetate, 4-isoproylcyclo-hexylmethyl acetate, linalyl acetate, lavandulyl acetate, citronellyl acetate, ox-terpinyl acetate, nopyl acetate, isobornyl acetate, bornyl acetate, isobornyl acetate, guaiyl acetate, 2-tert-butylyclohexyl acetate, 4-tert-butylcyclohexyl acetate, decahydro-β-naphthyl acetate, menthyl acetate, p-menthanyl acetate, neryl acetate, cinnamyl acetate, ethyl propionate, ethyl butyrate, butyl butyrate, isoamyl butyrate, hexyl butyrate, cis-3-hexenyl butyrate, cis-3-hexenyl isobutyrate, ethyl isovalerate, 2-methylbutyrate, ethyl hexanoate, 2-propenyl hexanoate, ethyl heptanoate, 2-propenyl heptanoate, ethyl octanoate, ethyl 2-trans-4-cis-decadienoate, methyl 2-nonynoate, benzyl propionate, benzyl isovalerate, phenylethyl isobutyrate, phenylethyl isovalerate, (α,α-dimethyl phenylethyl butyrate, methyl benzoate, hexyl benzoate, benzyl benzoate, ethyl phenylacetate, geranyl phenylacetate, 1-phenylethyl phenylacetate, methyl cinnamate, benzyl cinnamate, phenylethyl cinnamate, geranyl propionate, geranyl isobutyrate, geranyl isovalerate, linalyl propionate, linalyl buryrate, linalyl isobutyrate, citronellyl propionate, citronellyl isobutyrate, citronellyl isovalerate, citronellyl tiglate, allyl 3-cyclohexylpropionate, methyl dihydrojasmonate, methyl 2-hexyl-3-oxocyclopentane-carboxylate, and mixtures thereof.

Non-limiting examples of aldehydes which are suitable for use as fragrance raw materials or which are suitably released by the hydrolysis of the pro-fragrances or pro-accords of the present invention include phenylacetaldehyde, p-methyl phenylacetaldehyde, p-isopropyl phenylacetaldehyde, methylnonyl acetaldehyde, phenylpropanal, 3-(4-t-butylphenyl)-2-methyl propanal (Lilial), 3-(4-t-butylphenyl)-propanal (Bourgeonal), 3-(4-methoxyphenyl)-2-methylpropanal (Canthoxal), 3-(4-isopropylphenyl)-2-methylpropanal (Cymal), 3-(3,4-methylenedioxyphenyl)-2-methylpropanal (Helional), 3-(4-ethylpheny)-2,2-dimethylpropanal (Floralozone), phenylbutanal, 3-methyl-5-phenylpentanal, hexanal, trans-2-hexenal, cis-hex-3-enal, heptanal, cis-4-heptenal, 2-ethyl-2-heptenal, 2,6-dimethyl-5-heptenal (Melonal), 2,4-heptadienal, octanal, 2-octenal, 3,7-dimethyloctanal, 3,7-dimethyl-2,6-octadien-1-al, 3,7-dimethyl-1,6-octadien-3-al, 3,7-dimethyl-6-octenal (citronellal), 3, 7-dimethyl-7-hydroxyoctan-1-al (hydroxy citronellal), nonanal, 6-nonenal, 2,4-nonadienal, 2,6-nonadienal, decanal, 2-methyl decanal, 4-decenal, 9-decenal, 2,4-decadienal, undecanal, 2-methyldecanal, 2-methylundecanal, 2,6,10-trimethyl-9-undecenal (Adoxal), undec-10-enyl aldehyde, undec-8-enanal, dodecanal, tridecanal, tetradecanal, anisaldehyde, bourgenonal, cinnamic aldehyde, α-amylcinnam-aldehyde, α-hexyl cinnamaldehyde, methoxy-cinnamaldehyde, isocyclocitral, citronellyl oxyacet-aldehyde, cortexaldehyde, cumminic aldehyde, cyclamen aldehyde, florhydral, heliotropin, hydrotropic aldehyde, vanillin, ethyl vanillin, benzaldehyde, p-methyl benzaldehyde, 3,4-dimethoxybenzaldehyde, 3-and 4-(4-hydroxy-4-methyl-pentyl)-3-cyclohexene-1-carboxaldehyde (Lyral), 2,4-dimethyl-3-cyclohexene-1-carboxaldehyde (Triplal), 1-methyl-3-(4-methylpentyl)-3-cyclohexencarboxaldehyde (Vernaldehyde), p-methylphenoxyacetaldehyde (Xi aldehyde), and mixtures thereof.

Non-imiting examples of ketones which are suitable for use as fragrance raw materials or which are suitably released by the hydrolysis of the pro-fragrances or pro-accords of the present invention include α-damascone, β-damascone, δ-damascone, β-damascenone, muscone, 6,7-dihydro-1,1,2,3,3-pentamethyl-4(5H)-indanone (cashmeran), cis-jasmone, dihydroj asmone, α-ionone, β-ionone, dihydro-β-ionone, γ-methyl ionone, α-iso-methyl ionone, 4-(3, 4-methylenedioxyphenyl)butan-2-one, 4-(4-hydroxyphenyl)butan-2-one, methyl β-naphthyl ketone, methyl cedryl ketone, 6-acetyl-1,1,2,4,4,7-hexamethyltetralin (tonalid), 1-carvone, 5-cyclohexadecen-l-one, acetophenone, decatone, 2-[2-(4-methyl-3-cyclohexenyl-1-yl) propyl]cyclopentan-2-one, 2-sec-butylcyclohexanone, β-dihydro ionone, allyl ionone, α-irone, α-cetone, α-irisone, acetanisole, geranyl acetone, 1-(2-methyl-5-isopropyl-2-cyclohexenyl)-1-propanone, acetyl diisoamylene, methyl cyclocitrone, 4-t-pentyl cyclohexanone, p-t-butylcyclohexanone, o-t-butylcyclohexanone, ethyl amyl ketone, ethyl pentyl ketone, menthone, methyl-7,3-dihydro-2H-1,5-benzodioxepine-3-one, fenchone, and mixtures thereof.

Pro-fragrances and Pro-accords

In addition to fragrance raw materials, pro-fragrances and pro-accords may be suitably delivered by the copolymer carriers of the present invention. The use of pro-fragrances and pro-accords by the formulator can enhance the enduring nature of a fragrance delivered to a situs.

The following are non-limiting examples of pro-fragrances and pro-accords.

i) Orthoesters:

Orthoesters having the general formula:

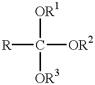

wherein hydrolysis of the orthoester releases fragrance raw material components according to the following scheme:

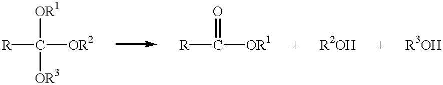

wherein the ester and alcohols are fragrance raw materials.

ii) Acetals:

Acetals having the general formula:

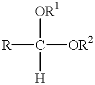

wherein hydrolysis of the acetal releases fragrance raw material components according to the following scheme:

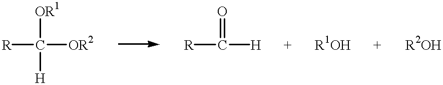

wherein the aldehyde and alcohols are fragrance raw materials.

iii) Ketals:

Ketals having the general formula:

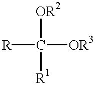

wherein hydrolysis of the ketal releases fragrance raw material components according to the following scheme:

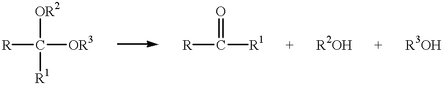

wherein the ketone and alcohols are fragrance raw materials.

iv) β-Ketoesters:

β-Ketoesters having the general formula:

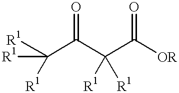

wherein hydrolysis of the ,-ketoesters releases a fragrance raw material alcohol ROH.

Other non-limiting examples of pro-fragrances and pro-accords include β-amino ketones, oxazolidines, tertahydro-1,3-oxazines, thiazolidines, tetrahydro-1,3-thiazines, orthocarbonates, and mixtures thereof.

Non-limiting examples of orthoester pro-fragrances or pro-accords according to the present invention are tris-geranyl orthoformate, tris(cis-3-hexen-1-yl) orthoformrate, tris(phenylethyl) orthoformate, bis(citronellyl) ethyl orthoacetate, tris(citronellyl) orthoformate, tris(cis-6-nonenyl) orthoformate, tris(phenoxyethyl) orthoformate, tris(geranyl, neryl) orthoformate (70:30 geranyl:neryl), tris(9-decenyl) orthoformate, tris(3-methyl-5-phenylpentanyl) orthoformate, tris(6-methylheptan-2-yl) orthoformate, tris([4-(2,2, 6-trimethyl-2-cyclohexen-1-yl)-3-buten-2-yl] orthoformate, tris[3-methyl-5-(2,2, 3-trimethyl-3-cyclopenten-1-yl)-4-penten-2-yl] penorthoformate, trismenthyl orthoformate, tris(4-isopropylcyclohexylethyl-2-yl) orthoformate, tris-(6,8-dimethylnonan-2-yl) orthoformate, tris-phenylethyl orthoacetate, tris(cis-3-hexen-1-yl) orthoacetate, tris(cis-6-nonenyl) orthoacetate, tris-citronellyl orthoacetate, bis(geranyl) benzyl orthoacetate, tris(geranyl) orthoacetate, tris(4-isopropylcyclohexylmethyl) orthoacetate, tris(benzyl) orthoacetate, tris(2,6-dimethyl-5-heptenyl) orthoacetate, bis(cis-3-hexen-1-yl) amyl orthoacetate, and neryl citronellyl ethyl orthobutyrate.

A description of pro-fragrances and pro-accords suitable for use in the systems of the present invention are encompassed in U.S. Pat. No. 5,710,122 Sivik et al., issued Jan. 20, 1998; U.S. Pat. No. 5,744,435 Hartman et al., issued Apr. 25, 1998; U.S. Pat. No. 5,756,827 Sivik, issued May 26, 1998; U.S. Pat. No. 5,919,752 Morelli et al., issued Jul. 6, 1999; U.S. Pat. N0. 5,958,870 Declercq et al., issued Sep. 28, 1999; U.S. Pat. No. 6,013,618 Morelli et al., issued Jan. 11, 2000; all of which are incorporated herein by reference. Suitable means for preparing ,-ketoesters is found in U.S. Pat. No. 5,965,767 Sivik et al., issued Oct. 12, 1999 incorporated herein by reference.

### Fragrance Delivery Systems

The fragrance delivery systems according to the present invention comprise:

A) an anaerobically biodegradable copolymer component, and

B) a fragrance component.

However, there are many embodiments or methods for delivering fragrance raw materials to a situs which can utilize the surprising discoveries of the present invention which are not related to laundry detergent compositions. For example, because the particles of the present invention are entrapped or entrained onto the material which comprises the situs, there is no requirement that the particle have a certain degree of substantivity, reactivity with, or affinity for the surface onto which they are deposited. This is unlike other carrier particles which can lead the formulator into a quagmire of balancing carrier affinity for the site of delivery with properties which allow for efficient and releasable absorption of the fragrance raw materials onto said particle. A single universal particle can sustain the delivery of all fragrance raw materials which a formulator may wish to deliver.

One embodiment of this universal particle relates to the fact the particles themselves are comprised of monomer which result in a “plastic,” semi-crystalline material having a hydrophobic portion into which the fragrance raw materials may partition. All things being equal and not wishing to be limited by theory, the release from a unit particle will be controlled by the partition coefficient of the fragrance raw material for the particle and the atmosphere. To this end the formulator may prepare particles each of which comprise a single fragrance raw material. The formulator may combine different particles and allow the fragrances to be released at the situs, thereby forming a perfume or fragrance accord as the fragrance raw materials are being released.

The formulator may also pre-form the accord and deliver it to a particle whereby each particle delivers the full perfume accord.

The following are non-limiting examples of fragrance components according to the present invention.

i) from about 0.1%, preferably from about 0.5%, more preferably from about 1%, yet more preferably from about 5%, most preferably from about 10% to about 100%, preferably to about 75%, more preferably to about 50% by weight, of at least one fragrance raw material, said fragrance raw material selected from the group consisting of primary, secondary, and tertiary alcohols, aldehydes, ketones, esters, ethers, terpenes, nitrites, and mixtures thereof; and

ii) the balance carriers and adjunct ingredients.

Another preferred fragrance component according to the present invention comprises:

i) from about 0.1%, preferably from about 0.5%, more preferably from about 1%, yet more preferably from about 5%, most preferably from about 10% to about 100%, preferably to about 75%, more preferably to about 50% by weight, of at least one fragrance raw material, said fragrance raw material selected from the group consisting of primary, secondary, and tertiary alcohols, aldehydes, ketones, esters, ethers, terpenes, nitrites, and mixtures thereof;

ii) one or more pro-fragrances or pro-accords selected from the group consisting of:

a) optionally from about 0.01%, preferably from about 0.5%, more preferably from about 5%, most preferably from about 25% to about 100%, more preferably to about 75%, most preferably to about 50% by weight, of an aldehyde or ketone releasing pro-fragrance or pro-accord as described herein above;

b) optionally from about 0.01%, preferably from about 0.5%, more preferably from about 5%, most preferably from about 25% to about 100%, more preferably to about 75%, most preferably to about 50% by weight, of an alcohol releasing pro-fragrance or pro-accord as described herein above;

c) optionally from about 0.01%, preferably from about 0.5%, more preferably from about 5%, most preferably from about 25% to about 99%, more preferably to about 75%, most preferably to about 50% by weight, of one or more pro-accords formed from at least one fragrance raw material, wherein said pro-accord is an orthoester;

d) and mixtures thereof; and

iii) the balance carriers and adjunct ingredients.

Another aspect of the present invention relates to “fragrance palate” delivery systems or “particle accords” wherein individual fragrance raw materials are delivered to the anaerobically biodegradable particles of the present invention. A series of single fragrance raw materials are applied to a anaerobically biodegradable polymers and maintained in separate containers. The formulator can then admix the desired fragrance accord by combining amounts of the individual particles. For fragrance raw materials which can be loaded at higher or lower levels, the formulator will then adjust the relative amount of the particle to be admixed.

A yet further aspect of the present invention relates to delivery of “fragrance families” via the particles of the present invention. For example, several fragrance raw materials which are typically found together, can be delivered as a fragrance family. Non-limiting examples of these fragrance characters are lemon, strawberry, bayberry, and the like. Typically these fragrance families comprise a fragrance raw material alcohol and one or more esters of said alcohol, inter alia, geraniol, geraniol acetate, geraniol formate.

This aspect of the present invention can be suitably defined as a “fragrance particle accord” comprising:

a) two or more fragrance raw material comprising anaerobically biodegradable particles according to the present invention wherein a single fragrance raw material is loaded onto each particle;

b) optionally particles which comprise pro-fragrances or pro-accords; and

c) optionally the balance particles which comprise adjunct ingredients.

### EMBODIMENTS OF THE PRESENT INVENTION

A preferred embodiment of the present invention is the delivery through the wash cycle of a particle to fabric which is capable of sustained release of a fragrance raw material or fragrance raw material accord. The following are examples of the use of the fragrance delivery systems of the present invention in the context of laundry detergent compositions.

Typically, laundry detergents comprise from about 0.01% to about 10% by weight, of fragrance raw materials depending upon the odor threshold values of the FRM'S. All things being equal, granular laundry detergent compositions, for example, will comprise from about 0.5% to about 2% by weight, of the composition FRM's.

The particles which comprise the fragrance delivery systems of the present invention which are adapted for use in laundry detergent compositions will typically comprise an amount of polymer or copolymer which is from about 2 times to about 10 times the weight of the fragrance or accord to be delivered. However, these ranges are not limiting and depending upon the embodiment and execution, the ranges may vary especially in light of the operable particle size range of the preferred embodiments.

The formulations of the present invention are not restricted to laundry compositions per se but can be any type of surfactant comprising cleaner, inter alia, rug shampoos, hard surface cleaners. Other formulations may be used to re-fresh fabric and will not comprise surfactants.

In the most basic form, the laundry detergent compositions of the present invention comprise:

A) from about 0.01% by weight, of a fragrance delivery system comprising:

a) from about 60% to about 99.9% by weight, of an anaerobically biodegradable particle having:

i) an average particle size of from about 5μ (microns), preferably from about 10μ, more preferably from about 35μ, most preferably from about 40μ to about 300μ, preferably to about 200μ, more preferably to about 100μ, most preferably to about 60μ;

ii) a density of from about 0.9 g/cm³ to about 1.5 glcm³, preferably about 1 g/cm³; and

b) from about 0.1% to about 40% by weight, of a fragrance or fragrance accord;

B) from about 0.01% by weight, preferably from about 0.1% more preferably from about 1% to about 60%, preferably to about 30% by weight, of said composition, of a surfactant system which comprises one or more surfactants selected from the group consisting of anionic, nonionic, cationic, zwitterionic, ampholytic surfactants, and mixtures thereof; and

C) the balance carriers and adjunct ingredients.

Example of formulations which benefit from the fragrance delivery systems of the present invention include formulations which comprise:

a) from about 0.01% by weight, of a fragrance delivery system as described herein above;

b) from about 0.5% to about 50% by weight, of a surfactant system comprising:

i) from about 10% to about 99% by weight, of said surfactant system, of a nonionic surfactant;

ii) from about 1% to about 90% by weight, of said surfactant system, of an anionic surfactant;

iii) optionally, from 1% to about 50% by weight, of said surfactant system, of a detersive surfactant selected from the group consisting of cationic surfactants, zwitterionic surfactants, ampholytic surfactants, and mixtures thereof; and

c) the balance carriers and adjunct ingredients.

Preferably the Heavy Duty Liquid (HDL) compositions according to the present invention comprise:

a) from about 0.01%, preferably from about 0.1%, more preferably from about 0.5%, most preferably form about 1% to about 10%, preferably to about 5%, more preferably to about 3%, most preferably to about 2% by weight, of a zwitterionic polyamine wherein said polyamine comprises more anionic substituents than the number of backbone quaternary nitrogen units;

b) from about 0.00005% to about 0.005% by weight, of an enzyme system comprising;

i) from about 20% to about 99% by weight, of a said enzyme system, a xyloglucanase enzyme;

ii) from about 1% to about 80% by weight, of said enzyme system, a mannanase enzyme;

iii) optionally, from 1% to 80% by weight, of said enzyme system one or more enzymes selected from the group consisting of protease enzymes, amylase enzymes, cellulase enzymes, lipolase enzymes, lipase enzymes, peroxidase enzymes, cutinase enzymes, and mixtures thereof;

c) from about 0.5% to about 50% by weight, of a surfactant system comprising:

i) from about 10% to about 99% by weight, of said surfactant system, of a nonionic surfactant;

ii) from about 1% to about 90% by weight, of said surfactant system, of an anionic surfactant;

iii) optionally, from 1% to about 50% by weight, of said surfactant system, of a detersive surfactant selected from the group consisting of cationic surfactants, zwitterionic surfactants, ampholytic surfactants, and mixtures thereof; and

d) the balance carriers and adjunct ingredients.

### SURFACTANT SYSTEM

The laundry detergent compositions of the present invention comprise a surfactant system. The surfactant systems of the present invention may comprise any type of detersive surfactant, non-limniting examples of which include one or more mid-chain branched alkyl sulfate surfactants, one or more mid-chain branched alkyl alkoxy sulfate surfactants, one or more mid-chain branched aryl sulfonate surfactants, one or more non mid-chain branched sulphonates, sulphates, cationic surfactants, zwitterionic surfactants, ampholytic surfactants, and mixtures thereof.

The total amount of surfactant present in the compositions of the present invention is from about 0.01% by weight, preferably from about 0.1% more preferably from about 1% to about 60%, preferably to about 30% by weight, of said composition.

Nonlimiting examples of surfactants useful herein include:

a) C₁₁-C₁₈ alkyl benzene sulfonates (LAS);

b) C₆-C₁₈ mid-chain branched aryl sulfonates (BLAS);

c) C₁₀-C₂₀ primary, α or ω-branched, and random alkyl sulfates (AS);

d) C₁₄-C₂₀ mid-chain branched alkyl sulfates (BAS);

e) C₁₀-C₁₈ secondary (2,3) alkyl sulfates as described in U.S. Pat. No. 3,234,258 Morris, issued Feb. 8, 1966; U.S. Pat. No. 5,075,041 Lutz, issued Dec. 24,1991; U.S. Pat. No. 5,349,101 Lutz et al., issued Sep. 20, 1994; and U.S. Pat. No. 5,389,277 Prieto, issued February 14, 1995 each incorporated herein by reference;

f) C₁₀-C₁₈ alkyl alkoxy sulfates (AEXS) wherein preferably x is from 1-7;

g) C₁₄-C₂₀ mid-chain branched alkyl alkoxy sulfates (BAEₓS);

h) C₁₀-C₁₈ alkyl alkoxy carboxylates preferably comprising 1-5 ethoxy units;

i) C₁₂-C₁₈ alkyl ethoxylates, C₆-C₁₂ alkyl phenol alkoxylates wherein the alkoxylate units are a mixture of ethyleneoxy and propyleneoxy units, C₁₂-C₁₈ alcohol and C₆-C₁₂ alkyl phenol condensates with ethylene oxide/propylene oxide block polymers inter alia Pluronic® ex BASF which are disclosed in U.S. Pat. No. 3,929,678 Laughlin et al., issued Dec. 30, 1975, incorporated herein by reference;

j) C₁₄-C₂₂ mid-chain branched alkyl alkoxylates, BAEₓ;

k) Alkylpolysaccharides as disclosed in U.S. Pat. No. 4,565,647 Llenado, issued Jan. 26, 1986, incorporated herein by reference;

l) Polyhydroxy fatty acid amides having the formula:

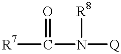

wherein R⁷ is C₅-C₃₁ alkyl; R⁸ is selected from the group consisting of hydrogen, C₁-C₄ alkyl, C₁-C₄ hydroxyalkyl, Q is a polyhydroxyalkyl moiety having a linear alkyl chain with at least 3 hydroxyls directly connected to the chain, or an alkoxylated derivative thereof; preferred alkoxy is ethoxy or propoxy, and mixtures thereof; preferred Q is derived from a reducing sugar in a reductive amination reaction, more preferably Q is a glycityl moiety; Q is more preferably selected from the group consisting of —CH₂(CHOH)ₙCH₂OH, —CH₂(CH₂OH)(CHOH)ₙ₋₁CH₂OH, —CH₂(CHOH)₂ —(CHOR′)(CHOH)CH₂OH, and alkoxylated derivatives thereof, wherein n is an integer from 3 to 5, inclusive, and R′ is hydrogen or a cyclic or aliphatic monosaccharide, which are described in U.S. Pat. No. 5,489,393 Connor et al., issued Feb. 6, 1996; and U.S. Pat. No. 5,45,982 Murch et al., issued Oct. 3, 1995, both incorporated herein by reference.

A non-limiting example of a nonionic surfactant suitable for use in the present invention has the formula:

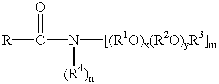

wherein R is C₇-C₂₁ linear alkyl, C₇-C₂₁ branched alky, C₇-C₂₁ linear alkenyl, C₇-C₂₁ branched alkenyl, and mixtures thereof.

R¹ is ethylene; R² is C₃-C₄ linear alkyl, C₃-C₄ branched alkyl, and mixtures thereof; preferably R² is 1,2-propylene.

R³ is hydrogen, C₁-C₄ linear alkyl, C₃-C₄ branched alkyl, and mixtures thereof; preferably hydrogen or methyl, more preferably hydrogen.

R⁴ is hydrogen, C₁-C₄ linear alkyl, C₃-C₄ branched alkyl, and mixtures thereof; preferably hydrogen. When the index m is equal to 2 the index n must be equal to 0 and the R⁴ unit is absent and is instead replaced by a -[(R¹O)ₓ(R²O)_{y}R³] unit.

The index m is 1 or 2, the index n is 0 or 1, provided that when m is equal to 1, n is equal to 1; and when m is 2 n is 0; preferably m is equal to 1 and n is equal to one, resulting in one-[(R¹O)ₓ(R²O)_{y}R³] unit and R⁴ being present on the nitrogen. The index x is from 0 to about 50, preferably from about 3 to about 25, more preferably from about 3 to about 10. The index y is from 0 to about 10, preferably 0, however when the index y is not equal to 0, y is from 1 to about 4. Preferably all of the alkyleneoxy units are ethyleneoxy units. Those skilled in the art of ethoxylated polyoxyalkylene alkyl amide surface active agents will recognized that the values for the indices x and y are average values and the true values may range over several values depending upon the process used to alkoxylate the amides.

The mid-chain branched alkyl sulfate surfactants of the present invention have the formula:

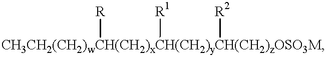

the alkyl alkoxy sulfates have the formula:

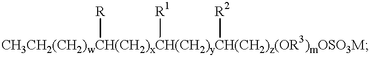

the alkyl alkoxylates have the formula:

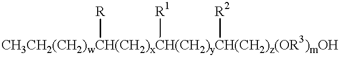

wherein R, R¹, and R² are each independently hydrogen, C₁-C₃ alkyl, and mixtures thereof; provided at least one of R, R¹, and R² is not hydrogen; preferably R, R¹, and R² are methyl; preferably one of R, R¹, and R² is methyl and the other units are hydrogen. The total number of carbon atoms in the mid-chain branched alkyl sulfate and alkyl alkoxy sulfate surfactants is from 14 to 20; the index w is an integer from 0 to 13; x is an integer from 0 to 13; y is an integer from 0 to 13; z is an integer of at least 1; provided w+x+y+z is from 8 to 14 and the total number of carbon atoms in a surfactant is from 14 to 20; R³ is C₁-C₄ linear or branched alkylene, preferably ethylene, 1,2-propylene, 1,3-propylene, 1,2-butylene, 1,4-butylene, and mixtures thereof. However, a preferred embodiment of the present invention comprises from 1 to 3 units wherein R³ is 1,2-propylene, 1,3-propylene, or mixtures thereof followed by the balance of the R³ units comprising ethylene units. Another preferred embodiment comprises R³ units which are randomly ethylene and 1,2-propylene units. The average value of the index m is at least about 0.01. When the index m has low values, the surfactant system comprises mostly alkyl sulfates with a small amount of alkyl alkoxy sulfate surfactant. Some tertiary carbon atoms may be present in the alkyl chain, however, this embodiment is not desired.

M denotes a cation, preferably hydrogen, a water soluble cation, and mixtures thereof. Non-limiting examples of water soluble cations include sodium, potassium, lithium, ammonium, alkyl ammonium, and mixtures thereof.

### ADJUNCT INGREDIENTS

The following are non-limiting examples of adjunct ingredients useful in the laundry compositions of the present invention, said adjunct ingredients include builders, optical brighteners, soil release polymers, dye transfer agents, dispersents, enzymes, suds suppressers, dyes, perfumes, colorants, filler salts, hydrotropes, photoactivators, fluorescers, fabric conditioners, hydrolyzable surfactants, preservatives, anti-oxidants, chelants, stabilizers, anti-shrinkage agents, anti-wrinkle agents, germicides, fungicides, anti corrosion agents, and mixtures thereof.

Builders -The laundry detergent compositions of the present invention preferably comprise one or more detergent builders or builder systems. When present, the compositions will typically comprise at least about 1% builder, preferably from about 5%, more preferably from about 10% to about 80%, preferably to about 50%, more preferably to about 30% by weight, of detergent builder.

Inorganic or P-containing detergent builders include, but are not limited to, the alkali metal, ammonium and alkanolammonium salts of polyphosphates (exemplified by the tripolyphosphates, pyrophosphates, and glassy polymeric meta-phosphates), phosphonates, phytic acid, silicates, carbonates (including bicarbonates and sesquicarbonates), sulphates, and aluminosilicates. However, non-phosphate builders are required in some locales. Importantly, the compositions herein function surprisingly well even in the presence of the so-called “weak” builders (as compared with phosphates) such as citrate, or in the so-called “underbuilt” situation that may occur with zeolite or layered silicate builders.

Examples of silicate builders are the alkali metal silicates, particularly those having a SiO₂:Na₂O ratio in the range 1.6:1 to 3.2:1 and layered silicates, such as the layered sodium silicates described in U.S. Pat. No. 4,664,839 Rieck, issued May 12, 1987. NaSKS-6 is the trademark for a crystalline layered silicate marketed by Hoechst (commonly abbreviated herein as “SKS-6”). Unlike zeolite builders, the Na SKS-6 silicate builder does not contain aluminum. NaSKS-6 has the delta-Na₂SiO₅ morphology form of layered silicate. It can be prepared by methods such as those described in German DE-A-3,417,649 and DE-A-3,742,043. SKS-6 is a highly preferred layered silicate for use herein, but other such layered silicates, such as those having the general formula NaMSiₓO_{2x+1y}H₂O wherein M is sodium or hydrogen, x is a number from 1.9 to 4, preferably 2, and y is a number from 0 to 20, preferably 0 can be used herein. Various other layered silicates from Hoechst include NaSKS-5, NaSKS-7 and NaSKS-11, as the alpha, beta and gamma forms. As noted above, the delta-Na₂SiO₅ (NaSKS-6 form) is most preferred for use herein. Other silicates may also be useful such as for example magnesium silicate, which can serve as a crispening agent in granular formulations, as a stabilizing agent for oxygen bleaches, and as a component of suds control systems.

Examples of carbonate builders are the alkaline earth and alkali metal carbonates as disclosed in German Patent Application No. 2,321,001 published on Nov. 15, 1973.

Aluminosilicate builders are useful in the present invention. Aluminosilicate builders are of great importance in most currently marketed heavy duty granular detergent compositions, and can also be a significant builder ingredient in liquid detergent formulations. Aluminosilicate builders include those having the empirical formula:

[M_{z}(zAlO₂)_{y}]xH₂O

wherein z and y are integers of at least 6, the molar ratio of z to y is in the range from 1.0 to about 0.5, and x is an integer from about 15 to about 264.

Useful aluminosilicate ion exchange materials are commercially available. These aluminosilicates can be crystalline or amorphous in structure and can be naturally-occurring aluminosilicates or synthetically derived. A method for producing aluminosilicate ion exchange materials is disclosed in U.S. Pat. No. 3,985,669, Krummel et al, issued Oct. 12, 1976. Preferred synthetic crystalline alurninosilicate ion exchange materials useful herein are available under the designations Zeolite A, Zeolite P (B), Zeolite MAP and Zeolite X. In an especially preferred embodiment, the crystalline aluminosilicate ion exchange material has the formula:

Na₁₂[(AlO₂)₁₂(SiO₂)₁₂] xH₂O

wherein x is from about 20 to about 30, especially about 27. This material is known as Zeolite A. Dehydrated zeolites (x=0-10) may also be used herein. Preferably, the aluminosilicate has a particle size of about 0.1-10 microns in diameter.

Organic detergent builders suitable for the purposes of the present invention include, but are not restricted to, a wide variety of polycarboxylate compounds. As used herein, “poly-carboxylate” refers to compounds having a plurality of carboxylate groups, preferably at least 3 carboxylates. Polycarboxylate builder can generally be added to the composition in acid form, but can also be added in the form of a neutralized salt. When utilized in salt form, alkali metals, such as sodium, potassium, and lithium, or alkanolammonium salts are preferred.

Included among the polycarboxylate builders are a variety of categories of useful materials. One important category of polycarboxylate builders encompasses the ether polycarboxylates, including oxydisuccinate, as disclosed in U.S. Pat. No. 3,128,287 Berg, issued Apr. 7, 1964, and U.S. Pat. No. 3,635,830 Lamberti et al., issued Jan. 18, 1972. See also “TMS/TDS” builders of U.S. Pat. No. 4,663,071 Bush et al., issued May 5, 1987. Suitable ether polycarboxylates also include cyclic compounds, particularly alicyclic compounds, such as those described in U.S. Pat. No. 3,923,679 Rapko, issued Dec. 2, 1975; U.S. Pat. No. 4,158,635 Crutchfield et al., issued Jun. 19, 1979; U.S. Pat. No. 4,120,874 Crutchfield et al., issued Oct. 17, 1978; and U.S. 4,102,903 Crutchfield et al., issued Jul. 25, 1978.

Other useful detergency builders include the ether hydroxypolycarboxylates, copolymers of maleic anhydride with ethylene or vinyl methyl ether, 1,3,5-trihydroxy benzene-2,4, 6-trisulphonic acid, and carboxymethyloxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, oxydisuccinic acid, polymaleic acid, benzene 1,3,5-tricarboxylic acid, carboxymethyloxysuccinic acid, and soluble salts thereof.

Citrate builders, e.g., citric acid and soluble salts thereof (particularly sodium salt), are polycarboxylate builders of particular importance for heavy duty liquid detergent formulations due to their availability from renewable resources and their biodegradability. Citrates can also be used in granular compositions, especially in combination with zeolite and/or layered silicate builders. Oxydisuccinates are also especially useful in such compositions and combinations.

Also suitable in the detergent compositions of the present invention are the 3,3-dicar-boxy4-oxa-1, 6-hexanedioates and the related compounds disclosed in U.S. Pat. No. 4,566,984, Bush, issued Jan. 28, 1986. Useful succinic acid builders include the C₅-C₂₀ alkyl and alkenyl succinic acids and salts thereof. A particularly preferred compound of this type is do-decenylsuccinic acid. Specific examples of succinate builders include: laurylsuccinate, myristylsuccinate, palmitylsuccinate, 2-dodecenylsuccinate (preferred), 2-pentadecenylsuccinate, and the like. Laurylsuccinates are the preferred builders of this group, and are described in European Patent Application 86200690.5/0,200,263, published Nov. 5, 1986.

Other suitable polycarboxylates are disclosed in U.S. Pat. No. 4,144,226, Crutchfield et al., issued Mar. 13, 1979 and in U.S. Pat. No. 3,308,067, Diehl, issued Mar. 7, 1967. See also Diehl U.S. Pat. No. 3,723,322.

Fatty acids, e.g., C₁₂-C₁₈ monocarboxylic acids, can also be incorporated into the compositions alone, or in combination with the aforesaid builders, especially citrate and/or the succinate builders, to provide additional builder activity. Such use of fatty acids will generally result in a diminution of sudsing, which should be taken into account by the formulator.

In situations where phosphorus-based builders can be used, and especially in the formulation of bars used for hand-laundering operations, the various alkali metal phosphates such as the well-known sodium tripolyphosphates, sodium pyrophosphate and sodium orthophosphate can be used. Phosphonate builders such as ethane-1-hydroxy-1,1-diphosphonate and other known phosphonates (see, for example, U.S. Pat. Nos. 3,159,581; 3,213,030; 3,422,021; 3,400,148 and 3,422,137) can also be used.

Dispersants When present, the laundry detergent compositions of the present invention comprise from about 0.01%, preferably from about 0.1%, more preferably from about 0.25%, most preferably from about 0.5% to about 5%, preferably to about 3%, more preferably to about 2% by weight, of one or more soil dispersants.

A description of suitable polyalkyleneimine dispersants which may be incorporated into the laundry detergent compositions of the present invention can be found in U.S. Pat. No. 4,597,898 Vander Meer, issued Jul. 1, 1986; European Patent Application 111,965 Oh and Gosselink, published Jun. 27, 1984; European Patent Application 111,984 Gosselink, published Jun. 27, 1984; European Patent Application 112,592 Gosselink, published Jul. 4, 1984; U.S. Pat. No. 4,548,744 Connor, issued Oct. 22, 1985; and U.S. Pat. No. 5,565,145 Watson et al., issued Oct. 15, 1996; all of which are included herein by reference. However, any suitable clay/soil dispersant or anti-redepostion agent can be used in the laundry compositions of the present invention.

In addition, polymeric dispersing agents which include polymeric polycarboxylates and polyethylene glycols, are suitable for use in the present invention. Polymeric polycarboxylate materials can be prepared by polymerizing or copolymerizing suitable unsaturated monomers, preferably in their acid form. Unsaturated monomeric acids that can be polymerized to form suitable polymeric polycarboxylates include acrylic acid, maleic acid (or maleic anhydride), fumaric acid, itaconic acid, aconitic acid, mesaconic acid, citraconic acid and methylenemalonic acid. The presence in the polymeric polycarboxylates herein or monomeric segments, containing no carboxylate radicals such as vinylmethyl ether, styrene, ethylene, etc. is suitable provided that such segments do not constitute more than about 40% by weight.

Particularly suitable polymeric polycarboxylates can be derived from acrylic acid. Such acrylic acid-based polymers which are useful herein are the water-soluble salts of polymerized acrylic acid. The average molecular weight of such polymers in the acid form preferably ranges from about 2,000 to 10,000, more preferably from about 4,000 to 7,000 and most preferably from about 4,000 to 5,000. Water-soluble salts of such acrylic acid polymers can include, for example, the alkali metal, ammonium and substituted ammonium salts. Soluble polymers of this type are known materials. Use of polyacrylates of this type in detergent compositions has been disclosed, for example, in U.S. Pat. No. 3,308,067 Diehl, issued Mar. 7, 1967.

Acrylic/maleic-based copolymers may also be used as a preferred component of the dispersing/anti-redeposition agent. Such materials include the water-soluble salts of copolymers of acrylic acid and maleic acid. The average molecular weight of such copolymers in the acid form preferably ranges from about 2,000, preferably from about 5,000, more preferably from about 7,000 to 100,000, more preferably to 75,000, most preferably to 65,000. The ratio of acrylate to maleate segments in such copolymers will generally range from about 30:1 to about 1:1, more preferably from about 10:1 to 2:1. Water-soluble salts of such acrylic acid/maleic acid copolymers can include, for example, the alkali metal, ammonium and substituted ammonium salts. Soluble acrylate/maleate copolymers of this type are known materials which are described in European Patent Application No. 66915, published Dec. 15, 1982, as well as in EP 193,360, published Sep. 3, 1986, which also describes such polymers comprising hydroxypropylacrylate. Still other useful dispersing agents include the maleic/acrylic/vinyl alcohol terpolymers. Such materials are also disclosed in EP 193,360, including, for example, the 45/45/10 terpolymer of acrylic/maleic/vinyl alcohol.

Another polymeric material which can be included is polyethylene glycol (PEG). PEG can exhibit dispersing agent performance as well as act as a clay soil removal-antiredeposition agent. Typical molecular weight ranges for these purposes range from about 500 to about 100,000, preferably from about 1,000 to about 50,000, more preferably from about 1,500 to about 10,000.

Polyaspartate and polyglutamate dispersing agents may also be used, especially in conjunction with zeolite builders. Dispersing agents such as polyaspartate preferably have a molecular weight (avg.) of about 10,000.

Soil Release Agents

The compositions according to the present invention may optionally comprise one or more soil release agents. If utilized, soil release agents will generally comprise from about 0.01%, preferably from about 0.1%, more preferably from about 0.2% to about 10%, preferably to about 5%, more preferably to about 3% by weight, of the composition. Polymeric soil release agents are characterized by having both hydrophilic segments, to hydrophilize the surface of hydrophobic fibers, such as polyester and nylon, and hydrophobic segments, to deposit upon hydrophobic fibers and remain adhered thereto through completion of the laundry cycle and, thus, serve as an anchor for the hydrophilic segments. This can enable stains occuring subsequent to treatment with the soil release agent to be more easily cleaned in later washing procedures.

The following, all included herein by reference, describe soil release polymers suitable for use in the present invention. U.S. Pat. No. 5,843,878 Gosselink et al., issued Dec. 1, 199; U.S. Pat. No. 5,834,412 Rohrbaugh et al., issued Nov. 10, 1998; U.S. Pat. No. 5,728,671 Rohrbaugh et al., issued Mar. 17, 1998; U.S. Pat. No. 5,691,298 Gosselink et al., issued Nov. 25, 1997; U.S. Pat. No. 5,599,782 Pan et al., issued Feb. 4, 1997; U.S. Pat. No. 5,415,807 Gosselink et al., issued May 16, 1995; U.S. Pat. No. 5,182,043 Morrall et al., issued Jan. 26, 1993; U.S. Pat. No. 4,956,447 Gosselink et al., issued Sep. 11, 1990; U.S. Pat. No. 4,976,879 Maldonado et al. issued Dec. 11, 1990; U.S. Pat. No. 4,968,451 Scheibel et al., issued Nov. 6, 1990; U.S. Pat. No. 4,925,577 Borcher, Sr. et al., issued May 15, 1990; U.S. Pat. No. 4,861,512 Gosselink, issued Aug. 29, 1989; U.S. Pat. No. 4,877,896 Maldonado et al., issued Oct. 31, 1989; U.S. Pat. No. 4,771,730 Gosselink et al., issued Oct. 27, 1987; U.S. Pat. No. 711,730 Gosselink et al., issued Dec. 8, 1987; U.S. Pat. No. 4,721,580 Gosselink issued Jan. 26, 1988; U.S. Pat. No. 4,000,093 Nicol et al., issued Dec. 28, 1976; U.S. Pat. No. 3,959,230 Hayes, issued May 25, 1976; U.S. Pat. No. 3,893,929 Basadur, issued Jul. 8, 1975; and European Patent Application 0 219 048, published Apr. 22, 1987 by Kud et al.

Further suitable soil release agents are described in U.S. Pat. No. 4,201,824 Voilland et al.; U.S. 4,240,918 Lagasse et al.; U.S. 4,525,524 Tung et al.; U.S. 4,579,681 Ruppert et al.; U.S. 4,220,918; U.S. Pat. No. 4,787,989; EP 279,134 A, 1988 to Rhone-Poulenc Chemie; EP 457,205 A to BASF (1991); and DE 2,335,044 to Unilever N.V., 1974; all incorporated herein by reference.

### METHOD OF USE

The present invention relates to a method of delivering fragrances to a situs, said method comprising the step of contacting a situs wherein an enhanced fragrance is desired with a composition comprising the fragrance delivery system of the present invention.

An example of a method according to the present invention comprises the step of contacting a situs with a composition according to the present invention.

The present invention further relates to the surprising discovery that delivery of a fragrance or perfume can be accomplished by selecting a particle having a particle size which nmatches the means or method by which the fragrance delivery systems is to be delivered. For example, the formulator who prepares a laundry detergent composition for use as a hand-wash composition will formulate a particle having an average particle size which is typically less than 100 microns. However, a particle to be delivered via an automatic laundry system will have an average particle size which is typically 100 microns or greater.

An example of a method according to the present invention comprises the steps of:

a) selecting an average particle size and forming a particle;

b) formulating a composition comprising said particle; and

c) contacting a situs with said composition.

The present invention also relates to a method for delivering a “fragrance particle accord”, said method comprising the steps of:

a) applying to a particle according to the present invention a single fragrance raw material;

b) combining two or more particles on to which is loaded different fragrance raw materials; and

c) applying said particles to a situs.

The following is a non-limiting example of a composition according to the present invention.

What is claimed is:

1. A fragrance raw material delivery system comprising:
A) anaerobically biodegradable particles having:
i) an average particle size sufficient to become entrapped on fabric;
ii) a density of from about 0.9 g/cm³ to about 1.5 g/cm³; and
B) a fragrance or fragrance accord.

2. A system according to claim 1 wherein said particle size is from about 5 microns.

3. A system according to claim 2 wherein said particle size is from about 10 microns to about 200 microns.

4. A system according to claim 3 wherein said particle size is from about 35 microns to about 100 microns.

5. A system according to claim 4 wherein said particle size is from about 40 microns to about 60 microns.

6. A system according to claim 1 wherein said particle has an average density of about 1 g/cm³.

7. A system according to claim 1 wherein said copolymer has a melt temperature, Tₘ, of from about 30 ° C. to about 160° C.

8. A system according to claim 7 wherein said Tm is from about 60° C. to about 140° C.

9. A system according to claim 8 wherein said Tm is from about 90° C. to about 120° C.

10. A system according to claim 1 wherein said particle comprises one or more monomer units selected form the group consisting of:
i) lower alkyl substituted units having the formula: 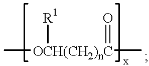
ii) higher alkyl substituted units having the formula: 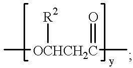
iii) and mixtures thereof; R¹ is hydrogen, C₁-C₂ alkyl, and mixtures thereof; n is 1 or 2; R² is C₃-C₁₉ alkyl, C₃-C₁₉ alkenyl, and mixtures thereof; wherein at least about 50% of said copolymer comprises lower alkyl substituted units; the indices x and y have values such that the copolymer has a weight average molecular weight of from 500 daltons to 5,000,000 daltons.

11. A system according to claim 10 wherein R¹ is methyl.

12. A system according to claim 10 wherein said copolymer further comprises one or more higher alkyl substituted units having the formula: 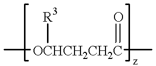
wherein R³ is C₃-C₁₉ alkyl, C₃-C₁₉ alkenyl, and mixtures thereof; R² does not equal R³; the index z has a value such that x+y+z provides a copolymer having a weight average molecular weight of from 500 daltons to 5,000,000 daltons.

13. A fragrance raw material delivery system comprising:
A) a biodegradable copolymer component, said component comprising monomer units selected from the group consisting of:
i) lower alkyl substituted units having the formula: 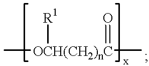
ii) higher alkyl substituted units having the formula: 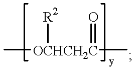
iii) and mixtures thereof; R¹ is hydrogen, C¹-C₂ alkyl, and mixtures thereof; n is 1 or 2; R² is C₃-C₁₉ alkyl, C₃-C₁₉ alkenyl, and mixtures thereof; wherein at least about 50% of said copolymer comprises lower alkyl substituted units; the indices x and y have values such that the copolymer has a weight average molecular weight of from 500 daltons to 5,000,000 daltons; and
B) a fragrance component, said fragrance component comprising ingredients selected from the group consisting of fragrance raw materials, pro-fragrances, pro-accords, and mixtures thereof.

14. A system according to claim 13 wherein said copolymer further comprises one or more higher alkyl substituted units having the formula: 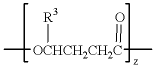
wherein R³ is C₃-C₁₉ alkyl, C₃-C₁₉ alkenyl, and mixtures thereof; the index z has a value such that x+y+z provides a copolymer having a molecular weight of from 500 daltons to 5,000,000 daltons.

15. A system according to claim 13 wherein R¹ is methyl.

16. A system according to claim 13 wherein said particle has an average particle size of from about 5 microns to about 300 microns.

17. A system according to claim 13 wherein said average particle size is from about 10 microns to about 200 microns.

18. A system according to claim 17 wherein said average particle size is from about 35 microns to about 100 microns.

19. A system according to claim 18 wherein said particle size is from about 40 microns to about 60 microns.

20. A system according to claim 13 wherein said particle has and an average density of from about 0.9 g/cm³ to about 1.5 g/cm³.

21. A system according to claim 20 wherein said particle has an average density of about 1 g/cm³.

22. A system according to claim 14 wherein said copolymer has a melt temperature, Tₘ, of from about 30° C. to about 160° C.

23. A system according to claim 22 wherein said Tm, is from about 60° C. to about 140° C.

24. A system according to claim 23 wherein said Tm is from about 90° C. to about 120° C.

25. A system according to claim 13 wherein said fragrance component comprises fragrance raw materials selected from the group consisting of primary, secondary, and tertiary alcohols, aldehydes, ketones, esters, carbonates, ethers, C₁₀-C₁₅ terpenes, and mixtures thereof.

26. A system according to claim 13 wherein said fragrance component comprises one or more pro-fragrances or pro-accords selected from the group consisting of acetals, ketals, orthoesters, β-ketoesters, β-amino ketones, oxazolidines, tertahydro-1,3-oxazines, thiazolidines, tetrahydro-1,3-thiazines, orthocarbonates, and mixtures thereof.

27. A system according to claim 13 wherein said fragrance component comprises:
i) from about 0.01% by weight, a fragrance raw material component comprising:
a) at least 0.01% by weight, of a mixture of one or more base note fragrances;
b) at least 0.01% by weight, of a mixture of one or more top or middle note fragrances;
c) the balance carriers, fixatives, and other adjunct ingredients; and
ii) optionally from about 0. 1% by weight, of a pro-fragrance or pro-accord component, said pro-fragrance or pro-accord selected from the group comprising:
a) from about 0.01% by weight, of one or more ketone or aldehyde releasing compounds;
b) from about 0.01% by weight, of one or more alcohol releasing compounds;
c) from about 0.01% by weight, of one or more ester releasing compounds;
d) from about 0.01% by weight, of one or more terpene releasing compounds; and
e) the balance carriers, stabilizers, and other adjunct ingredients.

28. A fragrance raw material delivery system comprising:
A) a biodegradable copolymer component, said component comprising monomer units selected from the group consisting of:
i) a lower alkyl substituted unit having the formula: 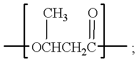
ii) higher alkyl substituted units having the formula: 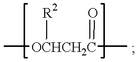
iii) and mixtures thereof; wherein R² is C₃-C₁₉ alky, C₃-C₁₉ alkenyl, and mixtures thereof; at least about 50% of said copolymer comprises said lower alkyl substituted unit; and
B) a fragrance component, said fragrance component comprising ingredients selected from the group consisting of fragrance raw materials, pro-fragrances, pro-accords, and mixtures thereof.

29. A laundry detergent composition comprising:
A) from about 0.01% by weight, of a fragrance delivery system comprising:
a) from about 60% to about 99.9% by weight, of an anaerobically biodegradable particle having:
i) a particle size of from about 5 microns to about 200 microns;
ii) a density of from about 0.9 g/cm³ to about 1.5 g/cm^{3,} preferably about 1 g/cm³; and
b) from about 01% to about 40% by weight, of a fragrance or fragrance accord;
B) from about 0.01% to about 60%by weight, of a surfactant system which comprises one or more surfactants selected from the group consisting of anionic, nonionic, cationic, zwitterionic, ampholytic surfactants, and mixtures thereof; and
C) the balance carriers and adjunct ingredients.

30. A laundry detergent composition comprising:
A) from about 0.01% by weight, of a fragrance delivery system comprising:
a) from about 60% to about 99.9% by weight, of an anaerobically biodegradable particle comprising:
i) lower alkyl substituted units having the formula: 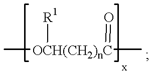
ii) higher alkyl substituted units having the formula: 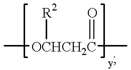
iii) and mixtures thereof; R¹ is hydrogen, C₁-C₂ alkyl, and mixtures thereof; n is 1 or 2; R² is C₃-C₁₉ alkyl, C₃-C₁₉ alkenyl, and mixtures thereof; wherein at least about 50% of said copolymer comprises lower alkyl substituted units; the indices x and y have values such that the copolymer has a molecular weight of from 500 daltons to 5,000,000 daltons; and
b) from about 0. 1 % to about 40% by weight, of a fragrance or fragrance accord;
B) from about 0.01% to about 60%by weight, of a surfactant system which comprises one or more surfactants selected from the group consisting of anionic, nonionic, cationic, zwitterionic, ampholytic surfactants, and mixtures thereof; and
C) the balance carriers and adjunct ingredients.

31. A fragrance particle accord comprising:
a) two or more fragrance raw material comprising an anaerobically biodegradable particle comprising:
i) lower alkyl substituted units having the formula: 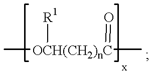
ii) higher alkyl substituted units having the formula: 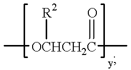
iii) and mixtures thereof; R¹ is hydrogen, C₁-C₂ alkyl, and mixtures thereof; n is 1 or 2; R² is C₃-C₁₉ alkyl, C₃-C₁₉ alkenyl, and mixtures thereof; wherein at least about 50% of said copolymer comprises lower alkyl substituted units; the indices x and y have values such that the copolymer has a molecular weight of from 500 daltons to 5,000,000 daltons;
wherein a single fragrance raw material is loaded onto each particle;
b) optionally particles which comprise pro-fragrances or pro-accords; and
c) optionally the balance particles which comprise adjunct ingredients.

32. A fragrance particle containing a fragrance family accord, said particle comprising:
a) two or more fragrance raw material comprising an anaerobically biodegradable particle comprising:
i) lower alkyl substituted units having the formula: 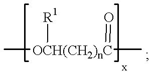
ii) higher alkyl substituted units having the formula: 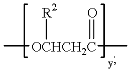
iii) and mixtures thereof;
R¹ is hydrogen, C₁-C₂ alkyl, and mixtures thereof; n is 1 or 2; R² is C₃-C₁₉ alkyl, C₃-C₁₉ alkenyl, and mixtures thereof; wherein at least about 50% of said copolymer comprises lower alkyl substituted units; the indices x and y have values such that the copolymer has a molecular weight of from 500 daltons to 5,000,000 daltons;
wherein two or more fragrance raw materials which comprise a fragrance family are loaded onto each particle;
b) optionally particles which comprise pro-fragrances or pro-accords; and
c) optionally the balance particles which comprise adjunct ingredients.

33. A method for providing enhanced fragrance to a situs comprising the steps of:
a) selecting an average particle size and forming an anaerobically biodegradable particle comprising:
i) lower alkyl substituted units having the formula: 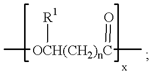
ii) higher alkyl substituted units having the formula: 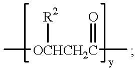
iii) and mixtures thereof;
R¹ is hydrogen, C₁-C₂ alkyl, and mixtures thereof; n is 1 or 2; R² is C₃-C₁₉ alkyl, C₃-C₁₉ alkenyl, and mixtures thereof; wherein at least about 50% of said copolymer comprises lower alkyl substituted units; the indices x and y have values such that the copolymer has a molecular weight of from 500 daltons to 5,000,000 daltons;
b) depositing a fragrance onto said particle; and
c) contacting a situs with said particle.

34. A method for providing enhanced fragrance to a situs comprising the steps of:
a) applying to an anaerobically biodegradable particle comprising:
i) lower alkyl substituted units having the formula: 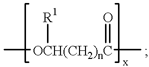
ii) higher alkyl substituted units having the formula: 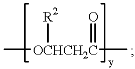
iii) and mixtures thereof; R¹ is hydrogen, C₁-C₂ alkyl, and mixtures thereof; n is 1 or 2; R² is C₃-C₁₉ alkyl, C₃-C₁₉ alkenyl, and mixtures thereof; wherein at least about 50% of said copolymer comprises lower alkyl substituted units; the indices x and y have values such that the copolymer has a molecular weight of from 500 daltons to 5,000,000 daltons; a single fragrance raw material;
b) admixing two or more particles onto which are loaded a different fragrance raw material; and
c) applying said particles to a situs.
